(19) United States
(12) Patent Application Publication
(10) Pub. No.: US 2025/0160809 A1
(43) Pub. Date: May 22, 2025

(54) SURGICAL RETRACTOR DEVICE AND METHOD OF USE

(71) Applicant: Nicholas J. Schoch, Shelby Township, MI (US)

(72) Inventor: Nicholas J. Schoch, Shelby Township, MI (US)

(21) Appl. No.: 18/955,935

(22) Filed: Nov. 21, 2024

Related U.S. Application Data
(60) Provisional application No. 63/629,712, filed on Nov. 21, 2023.

(51) Int. Cl. A61B 17/02; A61B 17/00

(52) U.S. Cl. CPC A61B 17/02; A61B 2017/0042

(57) ABSTRACT

The present disclosure relates in general to surgical instruments and methods of use. More specifically, but not exclusively, the present disclosure relates to surgical hard and soft tissue retractor devices and applications for use in orthopedic surgeries. The surgical retractor device of the present disclosure comprises a tip portion, an armature portion, and a handle portion that are designed to permit a surgeon to insert and maneuver an implant into position during surgery while reducing or eliminating surrounding tissue and/or nerve damage. While intended for use in reverse shoulder replacement surgery, it is contemplated by the present disclosure that the surgical retractor device may be universally applied across various surgical procedures that present challenges in highly constrained surgical space.

### CROSS-REFERENCE TO RELATED APPLICATIONS

This application claims priority under 35 U.S.C. § 119 to U.S. Provisional Patent Application No. 63/629,712 filed Nov. 21, 2023, herein incorporated by reference in its entirety.

### TECHNICAL FIELD

The present disclosure relates in general to surgical instruments and methods of use. More particularly, but not exclusively, the present disclosure relates to surgical hard and soft tissue retractor devices and applications for use in orthopedic surgeries. The purpose of the present disclosure is to provide improved control, retention, retraction, tissue preparation, tissue correction, and implant placement in or around hard and soft tissue during surgical procedures.

### BACKGROUND

Orthopedic surgeries, and particularly minimally invasive surgeries (“MIS”), provide limited access to the surgical site that is often complicated by surrounding hard and soft tissue. Indeed, surrounding hard and soft tissue must be reliably secured and controlled during surgery to provide for instrument placement and advancement, including adequate exposure to operative tissue to be repaired or replaced. Retractor devices traditionally utilized in the medical industry are specially adapted for certain procedures to provide a modicum of control of surrounding hard and soft tissue statically and/or dynamically. Because the structure of these traditional retractor devices are specialized, such devices cannot be universally applied across various surgical procedures that present challenges in highly constrained surgical space. Moreover, traditional retractor devices often fail to provide sufficient grip to surrounding hard and soft tissue without the device slipping out of position during surgery. Problems further persist with traditional retractor devices as they often fail to provide adequate spacing to permit a surgeon to insert and maneuver an implant into position during surgery.

As a result, a need exists for improved surgical retractor devices and methods that provide enhanced and reliable control of surrounding hard and soft tissue of the surgical site. A need further exists for surgical retractor devices and methods that may be universally applied across various surgical procedures that present challenges in highly constrained surgical space. A need still further exists for surgical retractor devices and methods that provide sufficient grip to surrounding hard and soft tissue to mitigate risk of the device slipping out of position during surgery. Another need exists for surgical retractor devices and methods that provide adequate spacing to permit a surgeon to insert and maneuver an implant into position during surgery while reducing or eliminating surrounding tissue damage. Yet another need exists for surgical retractor devices and methods that are simple to operate to minimize training time needed by surgeons and surgical staff, along with reducing operating room preparation time before surgery.

### SUMMARY

In one aspect of the present disclosure, a surgical retractor device is provided. The surgical retractor device may comprise a tip portion, an armature portion, and a handle portion. The surgical retractor device may be designed to provide control of surrounding hard and/or soft tissue of a surgical site.

In another aspect of the present disclosure, a surgical retractor device is provided. The surgical retractor device may comprise a tip portion having a leading edge, a top surface, and a bottom surface. The surgical retractor device may further comprise an armature portion having a straight section, an angled section, and a curved section. The surgical retractor device may still further comprise a handle portion having a flat section and an optional ring section. The surgical retractor device may be designed to provide control of surrounding hard and/or soft tissue of a surgical site during reverse shoulder replacement surgery.

In yet another aspect of the present disclosure, a method of using a surgical retractor device is provided. The method may comprise providing a retractor device. The surgical retractor device may comprise a tip portion having a leading edge, a top surface, and a bottom surface. The surgical retractor device may further comprise an armature portion having a straight section, an angled section, and a curved section. The surgical retractor device may still further comprise a handle portion having a flat section and an optional ring section. The method may further comprise utilizing the surgical retractor device to provide control of surrounding hard and/or soft tissue of a surgical site during reverse shoulder replacement surgery.

### Objects, Features, and Advantages

It is a principal object, feature, and advantage of the present disclosure to overcome the aforementioned deficiencies in the art and provide improved surgical retractor devices and methods that provide enhanced and reliable control of surrounding hard and soft tissue of the surgical site.

Another object, feature, and advantage of the present disclosure is to provide surgical retractor devices and methods that may be universally applied across various surgical procedures that present challenges in highly constrained surgical space.

Yet another object, feature, and advantage of the present disclosure is to provide surgical retractor devices and methods that provide exceptional grip to surrounding hard and soft tissue to mitigate risk of the device slipping out of position during surgery.

A further object, feature, and advantage of the present disclosure is to provide surgical retractor devices and methods that provide better spacing to permit a surgeon to insert and maneuver an implant into position during surgery while reducing or eliminating surrounding tissue and/or nerve damage.

Another object, feature, and advantage of the present disclosure is to provide surgical retractor devices and methods that provide a surgeon with an improved ability to ream, cut, and drill joint surfaces for a more efficient placement of reconstructive implants during joint reconstruction.

Yet another object, feature, and advantage of the present disclosure is to provide surgical retractor devices and methods that are simple to operate to minimize training time needed by surgeons and surgical staff, along with reducing operating room preparation time before surgery.

A further object, feature, and advantage of the present disclosure is to provide surgical retractor devices and methods that are cost efficient to produce while maintaining structural integrity for long term use.

Other objects, features, or advantages of this disclosure will become apparent from the following detailed description and claims, taken in conjunction with the accompanying drawings that set forth, by way of illustration and example and without limitation, certain aspects of this disclosure. No single aspect need provide each and every object, feature, or advantage. Thus the present disclosure is not to be limited to or by these objects, features, and advantages.

### BRIEF DESCRIPTION OF THE DRAWINGS

The accompanying drawings, incorporated herein and forming a part of the specification, illustrate aspects of the present disclosure together with the detailed description and claims.

FIG. 1 is an isometric view of a first aspect of the surgical retractor device of the present disclosure.

FIG. 2 is a top view of the surgical retractor device of FIG. 1.

FIG. 2A is the surgical retractor device of FIG. 2, further disclosing nonlimiting examples of dimension ranges.

FIG. 3 is a left side view of the surgical retractor device of FIG. 1, the right side view being a mirror image thereof.

FIG. 3A is the surgical retractor device of FIG. 3, further disclosing nonlimiting examples of dimension, curvature, and angle ranges.

FIG. 4 is front view of the surgical retractor device of FIG. 1.

FIG. 5 is an isometric view of a second aspect of the surgical retractor device of the present disclosure.

FIG. 6 is a top view of the surgical retractor device of FIG. 5.

FIG. 6A is the surgical retractor device of FIG. 6, further disclosing nonlimiting examples of dimension ranges.

FIG. 7 is a left side view of the surgical retractor device of FIG. 5, the right side view being a mirror image thereof.

FIG. 7A is the surgical retractor device of FIG. 7, further disclosing nonlimiting examples of dimension, curvature, and angle ranges.

FIG. 8 is front view of the surgical retractor device of FIG. 5.

FIG. 9 is an isometric view of a third aspect of the surgical retractor device of the present disclosure.

FIG. 10 is a top view of the surgical retractor device of FIG. 9.

FIG. 10A is the surgical retractor device of FIG. 10, further disclosing nonlimiting examples of dimension ranges.

FIG. 11 is a left side view of the surgical retractor device of FIG. 9, the right side view being a mirror image thereof.

FIG. 11A is the surgical retractor device of FIG. 11, further disclosing nonlimiting examples of dimension ranges.

FIG. 11B is a cross-sectional view of FIG. 11A, further disclosing nonlimiting examples of curvature ranges.

FIG. 12 is front view of the surgical retractor device of FIG. 9.

FIG. 13 is an isometric view of a fourth aspect of the surgical retractor device of the present disclosure.

FIG. 14 is a top view of the surgical retractor device of FIG. 13.

FIG. 14A is the surgical retractor device of FIG. 14, further disclosing nonlimiting examples of dimension ranges.

FIG. 15 is a left side view of the surgical retractor device of FIG. 13, the right side view being a mirror image thereof.

FIG. 15A is the surgical retractor device of FIG. 15, further disclosing nonlimiting examples of dimension ranges.

FIG. 15B is a cross-sectional view of FIG. 15A, further disclosing nonlimiting examples of curvature ranges.

FIG. 16 is front view of the surgical retractor device of FIG. 13.

### DETAILED DESCRIPTION

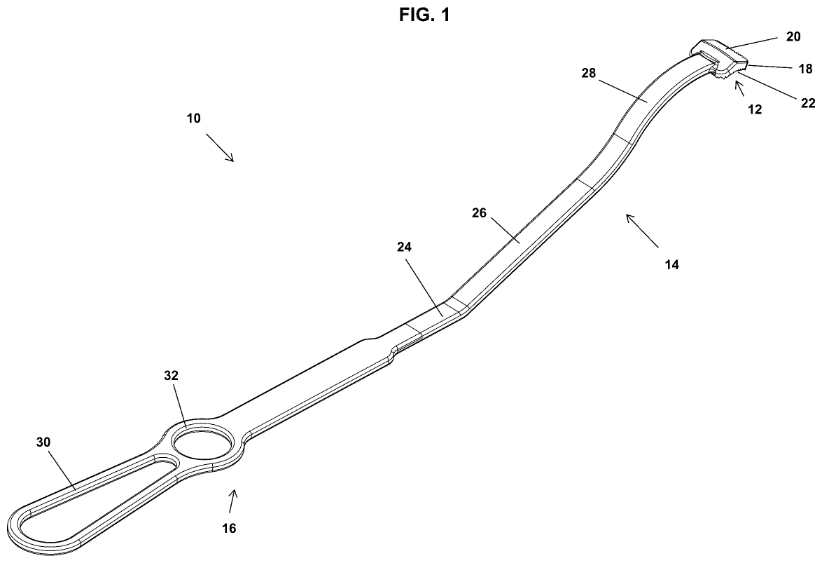

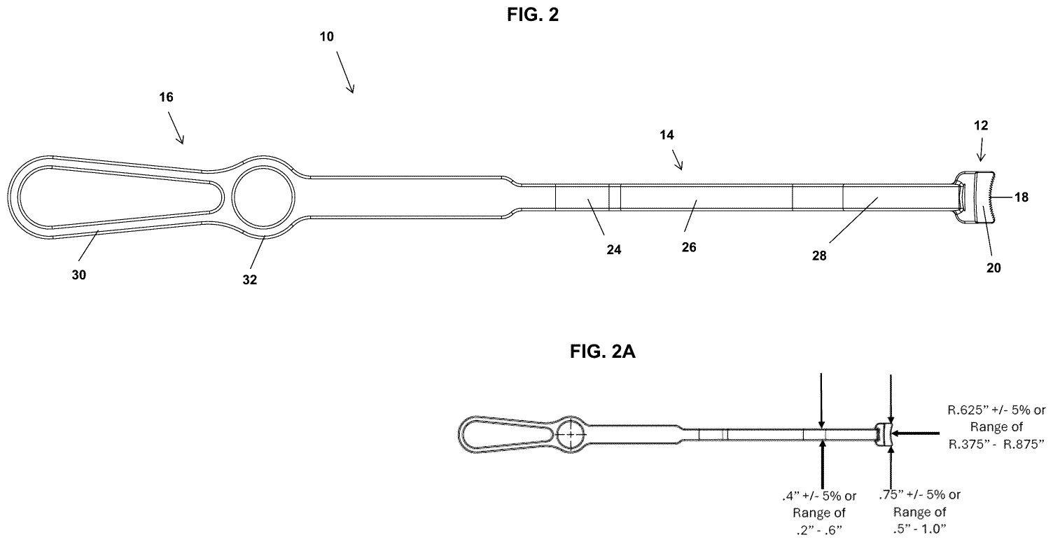

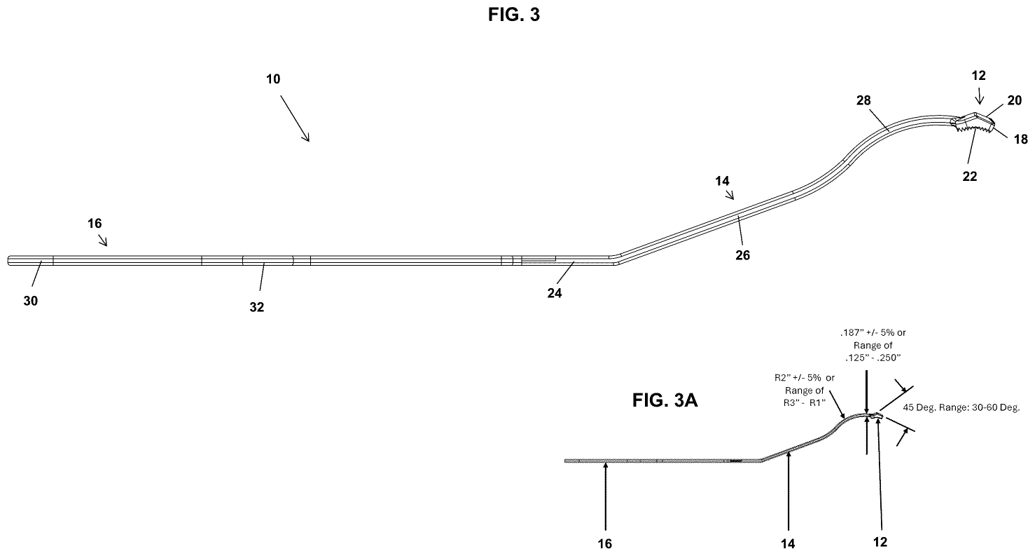

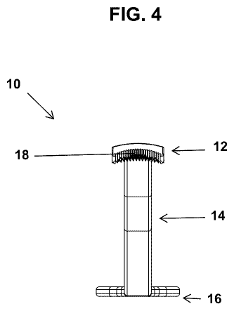

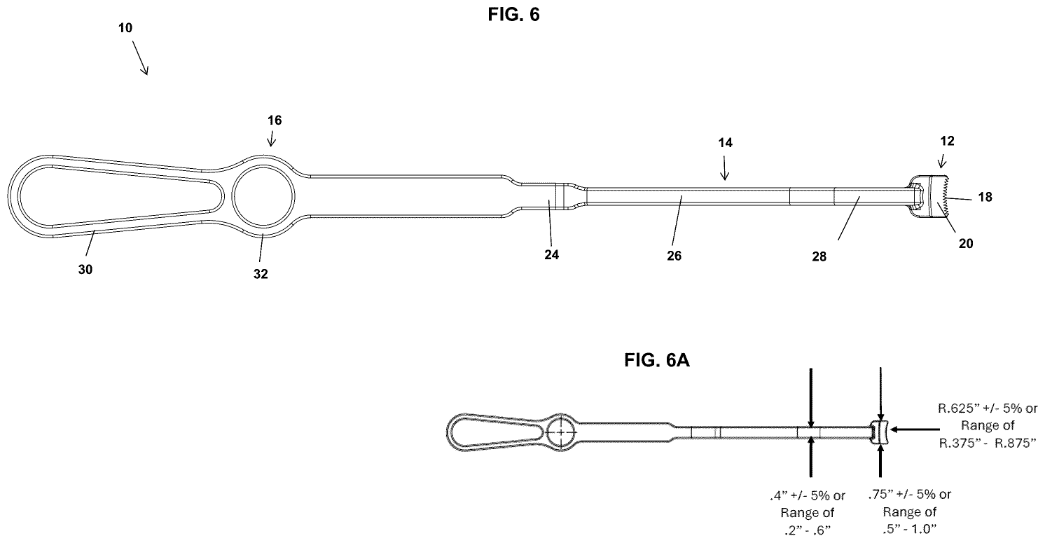

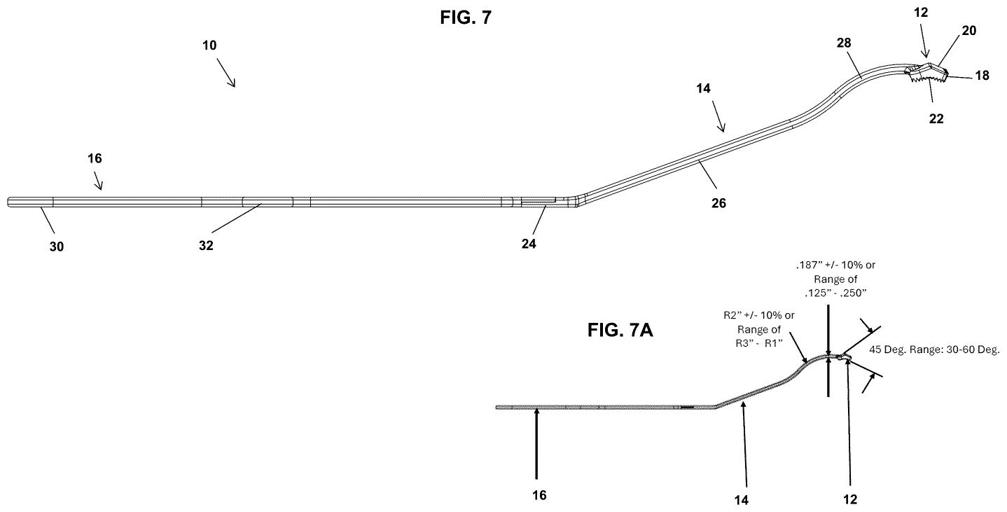

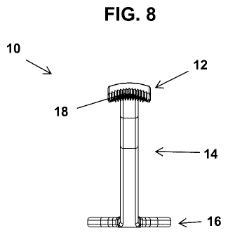

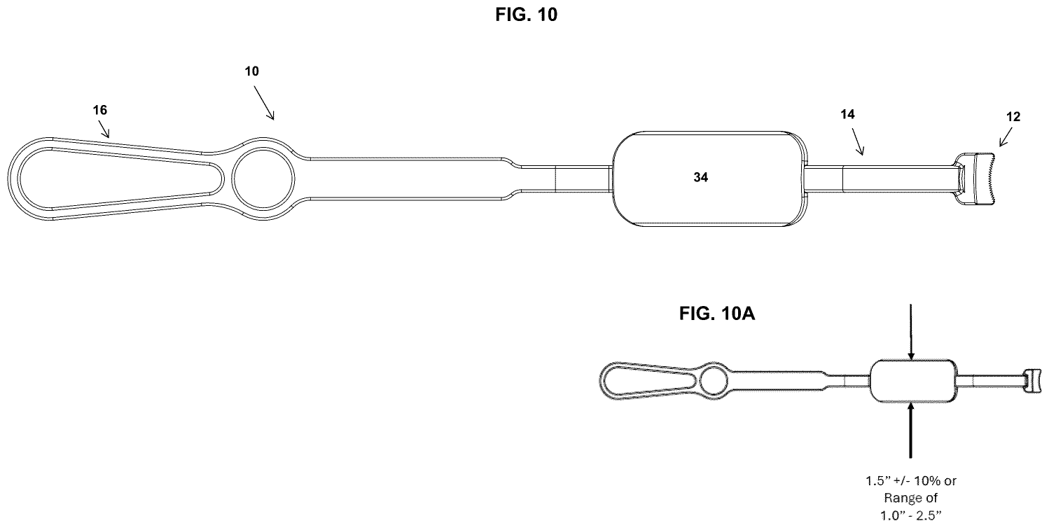

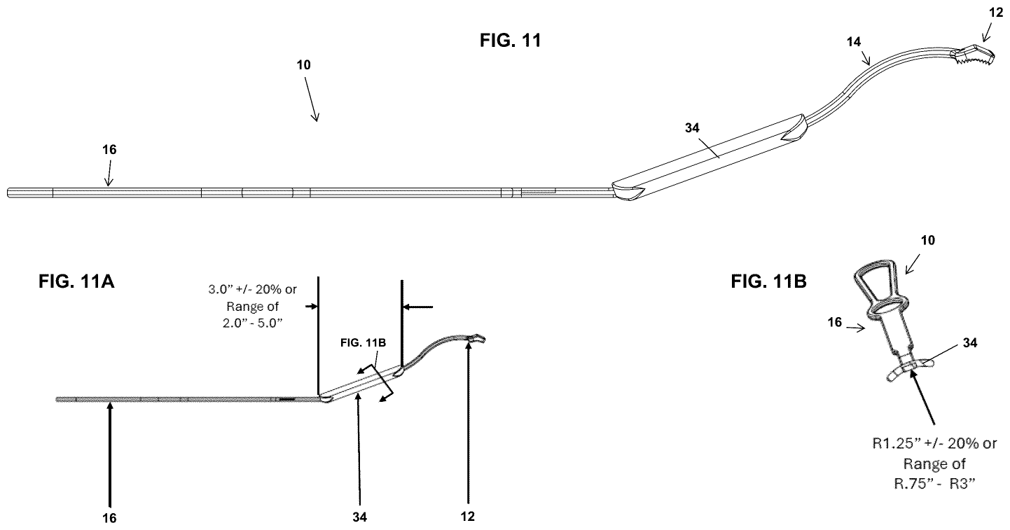

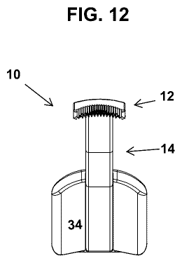

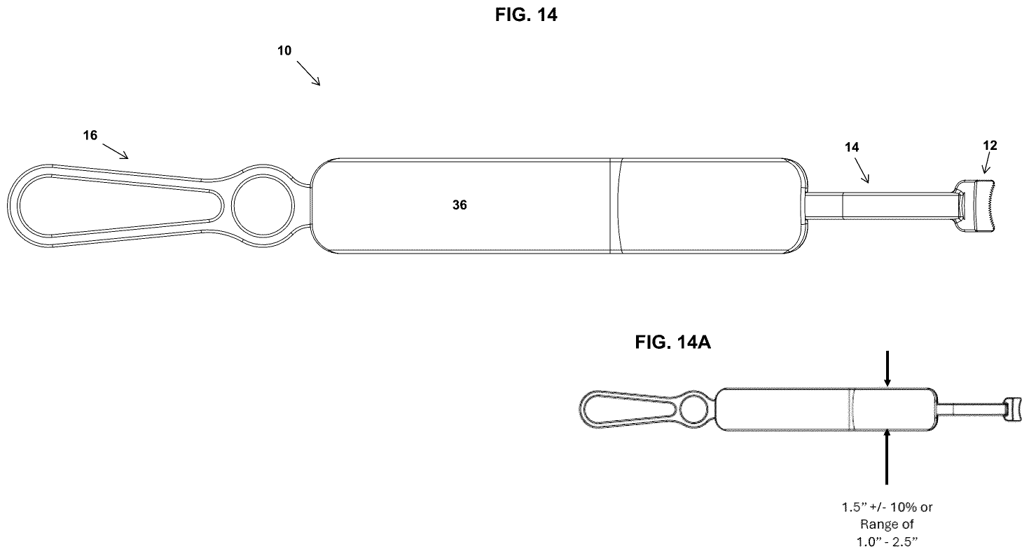

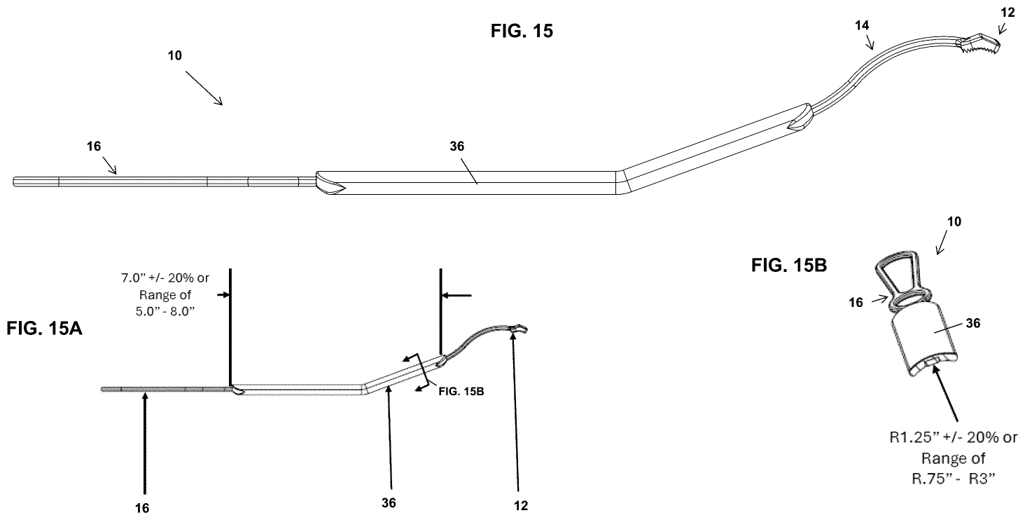

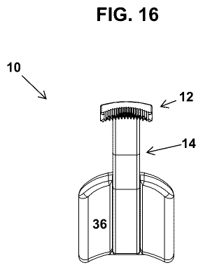

Referring generally to FIGS. 1-16, the present disclosure relates to surgical retractor devices and methods that provide enhanced and reliable control of surrounding hard and soft tissue of the surgical site. The surgical retractor device and methods of the present disclosure are designed to provide exceptional grip to surrounding hard and soft tissue to mitigate risk of the device slipping out of position during surgery. The surgical retractor device and methods of the present disclosure are further designed to provide better spacing to permit a surgeon to insert and maneuver an implant into position during surgery while reducing or eliminating surrounding tissue and/or nerve damage. While intended for human applications, nevertheless, the surgical retractor device and methods of the present disclosure may also be utilized in large or small animal veterinary applications.

While primarily intended for orthopedic MIS and standard surgical approaches involving reverse shoulder reconstruction, the surgical retractor device and methods of the present disclosure may also be utilized across various surgical procedures that present challenges in highly constrained surgical space. Orthopedic surgical examples where the present disclosure may be utilized include, but are not limited to, total and partial joint replacement surgeries. Other nonlimiting examples may include hip, knee, elbow, wrist, and ankle joint replacement surgeries.

While certain aspects of the present disclosure are shown and described herein, it is understood that such aspects are merely exemplary. The present disclosure is not intended to be limited to these specific aspects and may encompass other aspects or embodiments. Therefore, specific system and method details disclosed herein are not to be interpreted or inferred as limiting, but merely as a basis for the claims and as a representative basis for teaching one skilled in the art how to make and use the disclosed subject matter.

It must further be noted that the singular terms “a,” “an,” and “the” as used herein may include plural referents unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means.

All words of approximation as used in the present disclosure and claims should be construed to mean “approximate,” rather than “perfect” or “exact,” and may be used as a modifier to any other word, number, quantity, quality, value, or specified parameter. Words of approximation, include, but are not limited to terms such as “about,” “approximately,” “around,” “almost,” “generally,” “largely,” “essentially,” “substantially,” etc. As used herein, in some aspects, the terms “about” or “approximately” when preceding a numerical value may indicate the value plus or minus a range of 0.001, 0.002, 0.003, 0.004 or. 005 inches. In other aspects, the terms “about” or “approximately” when preceding a numerical value may indicate the value plus or minus a range of 0.01, 0.02, 0.03, 0.04 or 0.05 inches. In still further aspects, the terms “about” or “approximately” when preceding a numerical value may indicate the value plus or minus a range of 1, 2, 3, 4 or 5 degrees. In some aspects, the terms “about” or “approximately” when preceding a numerical value may indicate the value plus or minus a range of 0.001, 0.002, 0.003, 0.004 or 0.005 radians/inches (R #inches). In other aspects, the terms “about” or “approximately” when preceding a numerical value may indicate the value plus or minus a range of R.01, R.02, R.03, R.04 or R.05 inches.

Furthermore the transitional phrase “comprising” that is synonymous with “including,” “containing,” and “characterized by” as used herein is inclusive or open-ended and does not exclude additional, unrecited elements, steps or ingredients. Alternatively the transitional phrase “consisting of” as used herein is closed and excludes any element, step or ingredient not specified. The transitional phrase “consisting essentially of” limits the scope of a claim to the specified materials or steps and those that do not materially affect the basic and novel characteristics of the claims.

Illustrated in FIGS. 1-16, the surgical retractor device (10) may comprise different aspects that vary in size, thickness, and intended use depending on the surgical procedure. In some aspects, the surgical retractor device (10) may comprise an overall length ranging between approximately 7-16 inches. In other aspects, the surgical retractor device (10) may comprise an overall length ranging between approximately 11-14 inches. In further aspects, the surgical retractor device (10) may comprise an overall thickness ranging between approximately 0.125-0.250 inches. The overall length and thickness of the surgical retractor device (10) may be optimized for effective use during a particular surgical operation.

As a nonlimiting example, traditional retractor devices are often inadequate for reverse shoulder replacement surgery. Specifically, traditional retractor devices are often of an insufficient length and thickness to provide the necessary rigidity and leverage to retract the humerus, which requires significant force. Compounding this problem are patients with large shoulders, wherein traditional retractor devices are often too short and/or weak to be effective for reverse shoulder replacement surgery. The surgical retractor device (10) of the present disclosure overcomes these deficiencies by providing an optimal length to allow sufficient force and leverage to be applied to move the humerus to obtain exposure of the glenoid lip/edge. Moreover, the optimal thickness of the surgical retractor device (10) is important in maintaining rigidity of the device during retraction of the humerus to provide sufficient force, particularly, in patients with large shoulders.

Shown in FIGS. 1-16, the surgical retractor device (10) includes three primary regions that each comprise a specific geometrical shape designed to serve a specific purpose in improving: (1) control of surrounding hard and soft tissue of the surgical site; (2) spacing to insert and maneuver an implant into position; and (3) protection of surrounding tissue and/or nerves from damage during surgery. In particular, the three primary regions include a tip portion (12), connected to an armature portion (14), further connected to a handle portion (16). Each portion (12, 14, 16) of the surgical retractor device (10) when used in combination are designed to provide improved retraction functionality as compared to traditional retractor devices for yielding greater safety to the patient and efficiency for the surgeon during operations. Each portion (12, 14, 16) may be formed of medical grade stainless steel noncorrosive materials that have been heat and surface treated per industry standards. Each portion (12, 14, 16) may be formed separately and thereafter joined together during the manufacturing process (e.g., laser weld), or alternatively, formed integrally together during the manufacturing process to form the surgical retractor device (10).

Shown in FIGS. 1-16, the tip portion (12) may comprise a leading edge (18), an angled top surface (20), and a curved bottom surface (22). The tip portion (12) may comprise an overall width ranging between approximately 0.5-1.0 inches. The leading edge (18) may comprise a curvature ranging between approximately R.375-R.875 inches. The bottom surface (22) may comprise a curvature ranging between approximately R.125-R.875 inches. The leading edge (18) and the bottom surface (20) may include a series of raised ridges or knurls designed to assist with gripping soft and/or hard tissue during surgery. In some aspects, the top surface (20) may comprise an angle ranging between approximately 30-60°. In other aspects, the top surface (20) may comprise an angle of approximately 45°. The series of raised ridges/knurls and the particular angle of the top surface (20) are designed to help maintain the surgical retractor device (10) in a preferred position during surgery to prevent the tip portion (12) from skiving/moving medially and striking a sensitive neuro vascular bundle.

As a nonlimiting example, traditional retractor devices often fail to sufficiently move the humerus out of the way in reverse shoulder replacement surgery because such devices improperly sit on the back of the glenoid as opposed to the lip/edge. Traditional retractor devices also fail to provide a sufficient curvature where a surgeon may access and replace a glenoid component. Compounding these problems is when traditional retractor devices move during reaming, requiring a surgeon to reposition or replace the retractor during surgery.

The surgical retractor device (10) of the present disclosure overcomes these deficiencies because the tip portion (12) is designed to mate with the glenoid lip/edge at either a 5 or 7 o'clock position depending on whether surgery is performed on a right or left shoulder, respectively. The particular design of the tip portion (12) allows for a firm grip on the glenoid lip/edge, facilitating effective reaming and implant placement without the need for frequent repositioning during surgery. Moreover, the series of raised ridges/knurls on the leading edge (18) and the bottom curved surface (20) create several gripping surfaces which in turn prevent the slipping off of the posterior position on the glenoid lip/edge during implant insertion and placement. The particular angle of the top surface (20) does not have a tendency to slide down the back face of the glenoid, as opposed to traditional retractor devices, because the tip portion (12) is held on the glenoid lip/edge.

Shown in FIGS. 1-16, the armature portion (12) may comprise a straight section (24), an angled section (26), and a curved section (28). In some aspects, the armature portion (12) may have an overall length ranging between approximately 6-12 inches. In other aspects, the armature portion (12) may have an overall length ranging between approximately 6-8 inches. The overall width of the armature portion (12) may range between approximately 0.2-0.6 inches. The straight section (24) may form an angle of ranging between approximately 15-45° with the angled section (28). In some aspects, the curved section (26) may comprise a curvature ranging between approximately R1-R3 inches. In other aspects, the curved section (26) may comprise a curvature of approximately R1.5 inches.

In particular, the low profile of the armature portion (12) provides for deeper retraction with less blocking of precious surgical access space. This in turn causes less soft tissue contracture and resultant injury to surrounding hard and soft tissue, including nerves. Improved site exposure provided by the present disclosure further provides a surgeon with an increased ability to ream, cut, and drill during preparation of joint surfaces. This provides a more efficient placement of reconstructive implants during joint reconstruction. Moreover, the particular curvature in the curved section (28) is optimal in providing the surgeon an ability to secure the surgical retractor device (10) while remaining out of the way for better visibility and space to perform implantation of the implant.

As a nonlimiting example, the particular curvature of the curved section (28) of the armature portion (14) allows a surgeon to gain clearance to the glenoid in reverse shoulder replacement surgery to afford better exposure and space to perform required surgical interventions on the glenoid without removing or realignment due to the retractor abutting, hitting or damaging surrounding tissues. The curved section (28) of the armature portion (14) thus avoids and restricts damage to the surrounding soft tissues, namely muscles, ligaments, and neural tissues. The curved section's (28) specific geometry also appropriately loads hard bony tissue precisely to achieve the desired displacement. The particular curvature of the armature portion (14) is therefore designed to provide ample space for glenoid component placement, particularly in reverse shoulder replacements with fixed-angle, long posts.

Shown in FIGS. 1-16, the handle portion (16) may comprise an ergonomic flat section (30) and an optional ring section (32) to facilitate secure handling of the surgical retractor device (10) during operation (e.g., when using slippery gloves during surgery). In some aspects, the handle portion (16) may have an overall length ranging between approximately 6-9 inches. In other aspects, the handle portion (16) may have an overall length of approximately 7.5 inches. The overall width of the armature portion (12) may range between approximately 0.2-0.6 inches.

As a nonlimiting example, the handle portion (16) in coordination with the particular curvature of the armature portion (14) is designed to protect the deltoid muscle during reverse shoulder replacement surgery by preventing shredding during retraction. At the same time, the handle portion (16) aids in implant placement, reaming, and achieving excellent exposure of the glenoid lip/edge even when operating on patients with large shoulders.

Shown in FIGS. 1-4, is a first aspect of the surgical retractor device (10) of the present disclosure comprising a standard size (e.g., overall length ranging between approximately 12-16 inches).

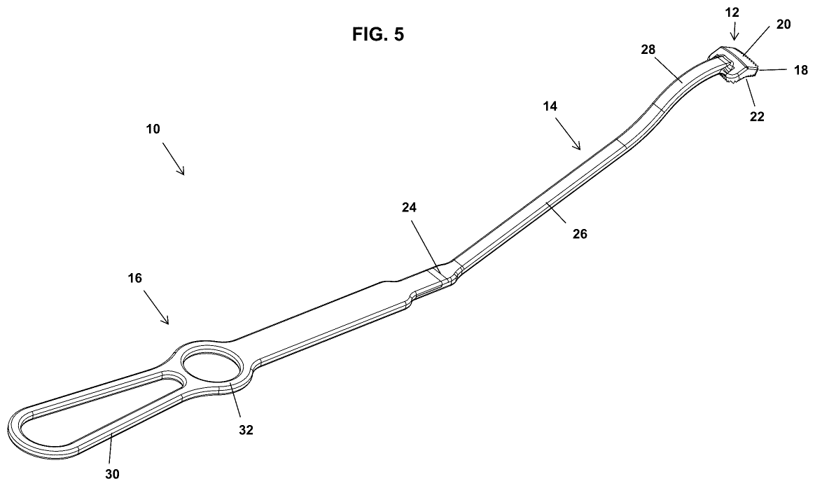

Shown in FIGS. 5-8, is a second aspect of the surgical retractor device (10) of the present disclosure comprising a small size (e.g., overall length ranging between approximately 7-11 inches).

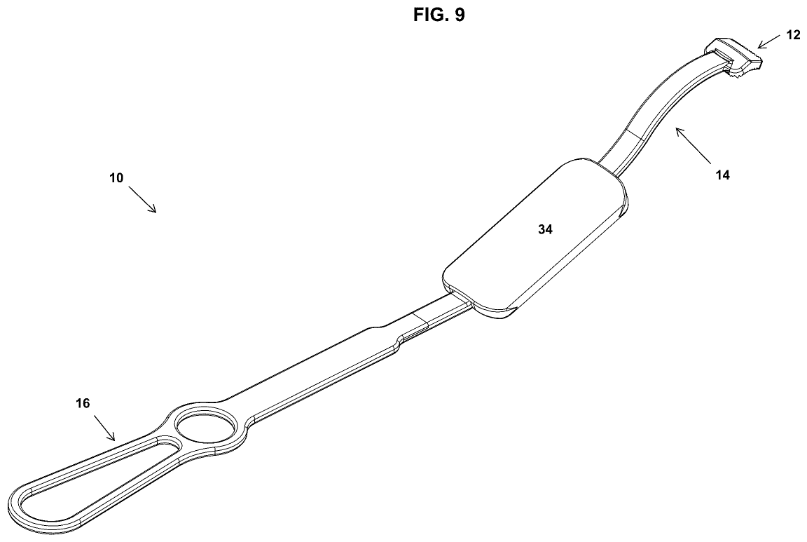

Shown in FIGS. 9-12, is a third aspect of the surgical retractor device (10) of the present disclosure comprising short deltoid cobra wings (34) on the armature portion (14) ranging between approximately 4-6 inches in length. The cobra wings (34) are designed to spread the force out and provide leverage during the surgical procedure to help diffuse the load and prevent surrounding hard and soft tissue damage during surgery. As a nonlimiting example, the cobra wings (34) are designed to allow displacement of the load against the deltoid muscle during reverse should replacement surgery.

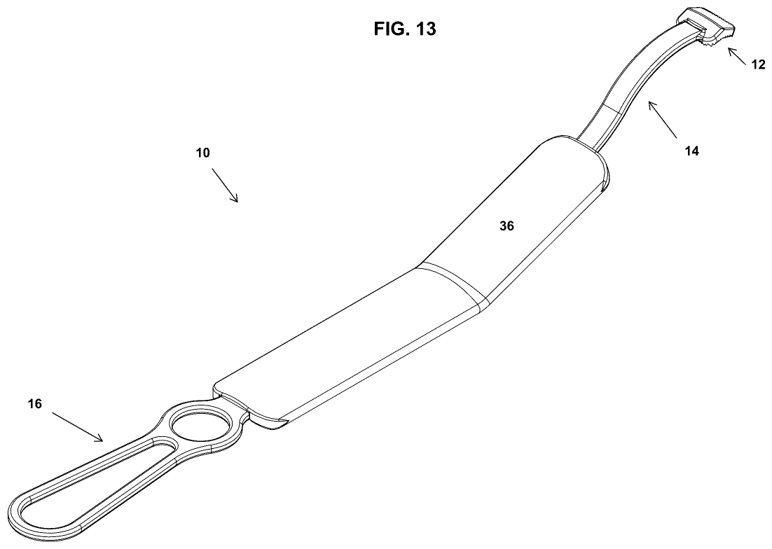

Shown in FIGS. 13-16, is a fourth aspect of the surgical retractor device (10) of the present disclosure comprising long deltoid cobra wings (36) on the armature portion (14) ranging between approximately 6-8 inches in length.

The particular combination of the tip portion (12), armature portion (14), and handle portion (16) of the surgical retractor device (10) of the present disclosure provides improved procedural options to a surgeon by allowing more joint space, better exposure, and mitigating ancillary tissue and nerve damage. As a nonlimiting example, the surgical retractor device (10) mitigates the risk of a damaged or paralyzed deltoid muscle during reverse shoulder replacement surgery which may result in an impaired or non-functional shoulder function and mobility. Indeed, the particular thickness and curvature of the surgical retractor device (10) protects the deltoid muscle from shredding while a surgeon is pulling retraction on it during surgery. At the same time the curvature allows implant placement and reaming while providing exceptional exposure even in patients with large shoulders. While intended for use in reverse shoulder replacement surgery, the present disclosure is not to be limited to the particular aspects and examples described herein and may be universally applied across various surgical procedures that present challenges in highly constrained surgical space.

The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of aspects, processes, or methods of the disclosure. It is understood that any other modifications, substitutions, and/or additions can be made, which are within the intended spirit and scope of the disclosure.

What is claimed is:

1: A retractor device, comprising:
a tip portion;
an armature portion; and
a handle portion;
wherein the retractor device is configured to provide control of surrounding hard and/or soft tissue of a surgical site.

2: The retractor device of claim 1, wherein the tip portion comprises a leading edge, a top surface, and a bottom surface.

3: The retractor device of claim 2, wherein the leading edge comprises a curvature ranging between approximately R.375-R.875 inches.

4: The retractor device of claim 2, wherein the bottom surface comprises a curvature ranging between approximately R.125-R.875 inches.

5: The retractor device of claim 2, wherein the leading edge and the bottom surface comprise a series of raised ridges or knurls.

6: The retractor device of claim 2, wherein the top surface comprises an angle ranging between approximately 30-60°.

7: The retractor device of claim 2, wherein the top surface comprises an angle of approximately 45°.

8: The retractor device of claim 1, wherein the armature portion comprises a straight section, an angled section, and a curved section.

9: The retractor device of claim 8, wherein the curved section comprises a curvature ranging between approximately R1-R3 inches.

10: The retractor device of claim 9, wherein the curved section comprises a curvature of approximately 1.5 inches.

11: The retractor device of claim 1, wherein the handle portion comprises a flat section and a ring section.

12: The retractor device of claim 1, wherein the armature portion includes deltoid cobra wings.

13: The retractor device of claim 1, wherein the retractor device is configured to provide control of surrounding hard and/or soft tissue of a surgical site during reverse shoulder replacement surgery.

14: A retractor device, comprising:
a tip portion, comprising:
a) a leading edge;
b) a top surface; and
c) a bottom surface;
an armature portion, comprising:
a) a straight section;
b) an angled section; and
c) a curved section;
a handle portion, comprising:
a) a flat section; and
b) an optional ring section;
wherein the retractor device is configured to provide control of surrounding hard and/or soft tissue of a surgical site during reverse shoulder replacement surgery.

15: The retractor device of claim 14, wherein the leading edge comprises a curvature ranging between approximately R.375-R.875 inches.

16: The retractor device of claim 14, wherein the bottom surface comprises a curvature ranging between approximately R.125-R.875 inches.

17: The retractor device of claim 14, wherein the leading edge and the bottom surface comprise a series of raised ridges or knurls.

18: The retractor device of claim 14, wherein the top surface comprises an angle ranging between approximately 30-60°.

19: The retractor device of claim 14, wherein the curved section comprises a curvature ranging between approximately R1-R3 inches.

20: A method of using a retractor device in reverse shoulder replacement surgery, comprising:
providing a retractor device, comprising:
a) a tip portion, comprising:
i. a leading edge;
ii. a top surface; and
iii. a bottom surface;
b) an armature portion, comprising:
i. a straight section;
ii. an angled section; and
iii. a curved section;
c) a handle portion, comprising:
i. a flat section; and
ii. an optional ring section;
utilizing the retractor device to provide control of surrounding hard and/or soft tissue of a surgical site during reverse shoulder replacement surgery.
